(19) United States
(12) Patent Application Publication
(10) Pub. No.: US 2003/0157090 A1
(43) Pub. Date: Aug. 21, 2003

(54) Stabilizing peptides, polypeptides and antibodies which include them

(76) Inventors: Eugeno Benvenuto, Roma (IT); Rosella Franconi, Anguillara Sabazia (IT); Angiola Desiderio, Anguillara Sabazia (IT); Paraskevi Tavladoraki, Roma (IT)

Correspondence Address: Nixon & Vanderhye, 8th Floor, 1100 North Glebe Road, Arlington, VA, 22201-4714, US

(21) Appl. No.: 10/169,351

(22) PCT Filed: Dec. 29, 2000
(86) PCT No.: PCT/IT00/00554

(30) Foreign Application Priority Data
Dec. 30, 1999 (IT) RM99 A 000803

(51) Int. Cl. A61K 39/395; C12Q 1/68; G01N 33/53; G01N 33/567; C07H 21/04; C07K 16/18; C12P 21/02; C12N 5/06

(52) U.S. Cl. 424/130.1; 435/6; 435/7.2; 435/69.1; 435/326; 435/320.1; 530/387.1; 536/23.53

(57) ABSTRACT

Object of the present invention are peptides which stabilize antibodies including them even in a reducing medium, in particular cytoplasmic medium, the polypeptides which include at least one of the above mentioned peptides, antibodies which include these polypeptides, and polynucleotides coding for peptides, polypeptides and antibodies of the invention.

### DESCRIPTION

1. Field of the Invention

The present invention relates to molecules capable of a specific interaction with target molecules.

2. State of the Art

Molecules capable of a specific interaction with targets are known in the art.

In particular antibodies are soluble and stable molecules known for being capable of an interaction with targets which is at the same time specific and effective.

Such molecules are used accordingly for modulating the accumulation and/or expression of target molecules in numerous applications in biology, medicine and agriculture.

It is generally desirable in these applications to have antibody with a great stability.

In particular there are applications wherein is necessary to have an antibody particularly stable wherein the interaction shall, or can, be carried out in an extremely reducing environment, such as the cytoplasm, for example applications carried out in order to:

block or stabilize interactions between macromolecules (for instance protein-protein or protein-DNA type);

modulate enzymatic functions covering the active site, confining the substrate or blocking an enzyme in its active or inactive structure;

remove proteins to their normal cellular compartment, for example confining transcription factors in the cytoplasm, or retaining membrane receptors in the endoplasmic reticulum;

interact with pathogen-derived molecules in order to interfere with the relevant infective process;

bind cytoplasmic molecules in vivo for diagnostic or therapeutic reasons (for instance oncogene products)

inside an intracellular compartment.

Among the antibodies known in the art as showing these features, scFv(F8), an engineered antibody in the scFv (“single chain variable fragment”) format, is one of the most representative (Tavladoraki et al 1993).

scFv(F8) in fact shows a significant molecular stability, as evidenced by measuring variations of free energy (ΔG) in denaturation and renaturation experiments in vitro (Tavladoraki et al. 1999).

This molecule is also characterized as having:

high efficiency in folding (Tavladoraki et al. 1999);

long half-life inside the cytoplasm (Tavladoraki et al. 1999);

functionality in the cytoplasm, in terms of ability to recognize antigens and interfere with the replication of the AMCV virus (Tavladoraki et al. 1993);

high levels of expression as soluble protein in bacterium and plant cytoplasm (Tavladoraki et al. 1999);

ability to interact in vivo with its antigen in the cytoplasm of eukaryotic cells, as shown by experiments carried out in the yeast “two-hybrid” system (Visintin et al. 1999).

Studies on the redox state of the protein, have further shown that no disulphide bond is formed in the protein inside the cytoplasm (Tavladoraki et al. 1999).

ScFv(F8) is a very interesting molecule for biotechnological applications, especially for those pointed out above.

scFv(F8) as such however, being capable of interacting with AMCV virus only, has a relatively limited field of application.

On the contrary, antibodies “scFv(F8)-like” (from the functional point of view) capable of interacting with targets other than AMCV, would be instead of great interest. They would enable a number of applications on specific targets in the cytoplasm or, given the stability, in other different environments.

### SUMMARY OF THE INVENTION

Object of the present invention are peptides able to confer stability and solubility to an antibody including them.

According to the present invention such an object is achieved by a peptide characterized in having a sequence selected from the group consisting of the sequences reported in the annexed sequence listing from SEQ ID NO: 1 to SEQ ID N: 8, and in that included in a variable region of a scFv antibody make said antibody soluble and stable in cytoplasm medium.

In order to make said antibody stable and soluble the peptides having the sequences SEQ ID NO: 1 (H-FR1), SEQ ID NO: 2, (H-FR2), SEQ ID NO: 3, (H-FR3), and SEQ ID NO: 4 (H-FR4), herein also denominated H-FR peptides, shall be included in the variable region of the heavy chain of said antibody (VH region), covalently linked to peptides having the sequences reported in the annexed sequence listing from SEQ ID NO:88 (H-CDR1), SEQ ID NO:89 (H-CDR2) and SEQ ID NO:90 (H-CDR3), herein also denominated H-CDR peptides, in the order

SEQ ID NO: 1 (H-FR1)-SEQ ID NO: 88-(H-CDR1)-SEQ ID NO: 2 (H-FR2)-SEQ ID NO: 89(H-CDR2)-SEQ ID NO: 3(H-FR3)-SEQ ID NO: 90 (H-CDR3)-SEQ ID NO: 4(H-FR4)

According to the arrangement shown in FIG. 1.

The peptides having the sequences SEQ ID NO: 5 (L-FR1), SEQ ID NO: 6, (L-FR2), SEQ ID NO: 7, (L-FR3), and SEQ ID NO: 8 (L-FR4), generally denominated L-FR peptides, shall instead be included in the variable region of the light chain of said antibody (VL region), covalently linked to peptides having the sequences reported in the annexed sequence listing from SEQ ID NO:91 (L-CDR1), SEQ ID NO:92 (L-CDR2) and SEQ ID NO:93 (L-CDR3), herein also denominated L-CDR peptides, in the order

SEQ ID NO: 5(L-FR1)-SEQ ID NO: 91-(L-CDR1)-SEQ ID NO: 6 (L-FR2)-SEQ ID NO: 92(L-CDR2)-SEQ ID NO: 7(L-FR3)-SEQ ID NO: 93 (L-CDR3)-SEQ ID NO: 8(L-FR4),

position 18 of H-FR3 (SEQ ID NO: 3) is Asn; Xaa in position 20 of H-FR3 (SEQ ID NO: 3) is Leu; Xaa in position 12 of H-FR7 (SEQ ID NO: 7) is Arg, Xaa in position 15 of H-FR7 (SEQ ID NO: 7) is Phe.

According to the invention all these peptides can be produced by means of a series of conventional techniques known in the art such as for example recombinant DNA but also constructions of synthetic polypeptides.

The relevant process for obtaining peptides stabilizing an antibody including them in a variable region comprising the steps of:

deriving a polypeptides from a monoclonal antibody,

mutagenizing said polypeptide by techniques, as hierarchical mutagenesis, error-prone PCR, DNA shuffling, or ribosome display,

is included in the object of the invention.

Alternatively said peptides can be derived by chemical synthesis, as for instance by translating a polynucleotide coding for them.

The H-CDR and L-CDR peptides of the invention are herein also denominated CDRs (Complementarity Determining Regions) peptides. CDRs peptides according to the present invention correspond to the parts of the regions VH and VL of the antibody which determine the binding specificity of the antibody. In particular H-CDRs peptides correspond to the parts of VH and L-CDRs peptides correspond to the parts of VL, which determine the binding specificity of the antibody.

The structural characteristics of the CDRs peptide are such that, when included in the polypeptides VH and VL covalently bound to the peptides FRs according to the above arrangement, they can give specificity to an antibody including them without modifying its stability and solubility.

Accordingly their sequences depend on the antigen that the antibody in which they are included specifically bind.

In order to derive polypeptides suitable as VH or VL region of an antibody can be used a process for deriving a polypeptide suitable as a variable region of an antibody and specific for a predetermined antigen comprising the step of:

producing an antibody having as a variable region of the heavy chain the polypeptide having the sequence reported in the sequence listing as SEQ ID NO: 101, and as a variable region of the light chain the polypeptide having the sequence reported in the annexed sequence listing as SEQ ID NO: 102;

putting in contact said antibody with said antigen and

selecting the antibody binding said antigen,

isolating the polypeptide of the variable region of the heavy chain and/or the polypeptide of the variable region of the light chain of the antibody binding said antigen.

This process, which is an object of the invention, in a preferred embodiment further comprises the step of

sequencing the variable regions of said antibody binding said antigen.

The predetermined antigen of the process of the invention can be any antigen of interest in particular the viral ones, and specifically the antigens associated to HIV, HCV and HPV, as Tat, Rev, E7 or NS3 protein, which shall be considered preferred. Other antigens like, bovine seroalbumin, lysozime, AMCV virus, “tomato spotted wilt virus” or “cucumber mosaic virus”, are used in a preferred embodiment of the invention.

Object of the invention are further the polypeptides obtainable by the above process, which are suitable for constituting the variable region of the heavy chain (VH) or the light chain (VL) of a given antibody.

Regarding the polypeptides of the invention, polypeptides suitable for constituting the VH region of an antibody, are also herein denominated VH polypeptides,

while polypeptides suitable for constituting the VL region of an antibody are also herein denominated VL polypeptides.

In particular in this regard the following polypeptides VH and VL polypeptides are an object of the present invention:

VH-F8 (SEQ ID NO: 31) and VL-F8 (SEQ ID NO: 32) which respectively constitute the VH and VL regions, of specific AMCV antibodies and in particular of scFv(F8);

VH-LYS/P5 (SEQ ID NO: 33) and VL-LYS/P5 (SEQ ID NO: 34) that respectively constitute the VH and VL regions, of antibodies specific for the lysozyme;

VH-LYS/11E (SEQ ID NO: 35) and VL-LYS/11E (SEQ ID NO: 36), which constitute the VH and VL regions respectively of specific antibodies for the lysozyme;

VH-BSA/9F (SEQ ID NO: 37) and VL-BSA/9F (SEQ ID NO:38) which constitute the VH and VL regions respectively of antibodies specific for bovine seroalbumin;

VH-TSWV(BR01)/6H (SEQ ID NO: 39) and VL-TSWV(BRO1)/6H(SEQ ID NO: 40), which constitute the VH and VL regions respectively of antibodies specific for the “tomato spotted wilt virus”;

VH-TSWV(P105)/1C (SEQ ID NO: 41) and VL-TSWV(P105)/1C (SEQ ID NO: 42) which constitute the VH and VL regions respectively of antibodies specific for the “tomato spotted wilt virus”;

VH-CMV/4G (SEQ ID NO: 43) and VL-CMV/4G(SEQ ID NO: 44), which constitute the VH and VL regions respectively of antibodies specific for the “cucumber mosaic virus”;

VH-CMV/4B (SEQ ID NO: 45) and VL-CMV/4B(SEQ ID NO: 46), which constitute the VH and VL regions respectively of antibodies specific for the “cucumber mosaic virus”; and

VH-CMV/2G (SEQ ID NO: 47) and VL-CMV/2G(SEQ ID NO: 48), which constitute the VH and VL regions respectively of antibodies specific for the “cucumber mosaic virus”.

On the basis of the polypeptides of the invention specific CDRs peptides can be derived giving binding specificity to the antibodies including them.

Said CDRs peptides can be derived by a process for deriving a peptide conferring to an antibody binding specificity to a predetermined antigen comprising the step of:

producing an antibody having as a variable region of the heavy chain the polypeptide having the sequence reported in the sequence listing as SEQ ID NO: 101, and as a variable region of the light chain the polypeptide having the sequence reported in the annexed sequence listing as SEQ ID NO: 102;

putting in contact said antibody with said antigen and

selecting the antibody binding said antigen,

isolating the polypeptide of the variable region of the heavy chain and/or the polypeptide of the variable region of the light chain of the antibody binding said antigen;

isolating from said polypeptide the part conferring binding specificity to said antibody. In a preferred embodiment the above process further comprises the step of

sequencing the variable regions of said antibody binding said antigen.

The antigen can be any antigen and preferably the ones described above.

Object of the present invention are also all the peptides obtainable by the above described process.

In particular the peptides CDRs which give the binding specificity for

ACMV (artichoke mottle crinkle virus) in the antibody scFv(F8), having respectively the sequences SEQ ID NO: 9 (H-CDR1-F8), SEQ ID NO: 10 (H-CDR2-F8), SEQ ID NO: 11 (H-CDR3-F8), SEQ ID NO: 12(L-CDR1-F8), SEQ ID NO: 13 (L-CDR2-F8), and SEQ ID NO: 14 (L-CDR3-F8);

lysozyme (in particular lysozyme of chicken egg albumen), with the sequences SEQ ID NO: 97 (H-CDR1-LYS/P5), SEQ ID NO: 98 (H-CDR2-LYS/P5), SEQ ID NO: 15 (H-CDR3-LYS/PS), SEQ ID NO: 99 (L-CDR1-LYS/P5), SEQ ID NO: 100 (L-CDR2-LYS/P5), and SEQ ID NO: 16 (L-CDR3-LYS/P5), are a further object of the present invention.

Object of the present invention are also all the peptides H-CDR3 and L-CDR3 as above defined which give binding specificity for antigens different from ACMV to the antibodies which include them in the VH and VL polypeptides substituting the original H-CDR3-F8 and L-CDR3-F8, together with other peptides H-CDR1, H-CDR2 and L-CDR2 of F8 and with the peptides L-CDR1-MUT (SEQ ID NO:76) included instead of the peptide L-CDR1-F8, according to the arrangement shown in FIG. 7.

Among these in particular object of the invention are the peptides H-CDR3 and L-CDR3 which give specificity for

lysozyme (H-CDR3-LYS/11E, SEQ ID NO: 17 and L-CDR3-LYS/11E, SEQ ID NO: 18),

bovine seroalbumin (H-CDR3-BSA/9F, SEQ ID NO: 19 and L-CDR3-BSA/9F, SEQ ID NO: 20),

“tomato spotted wilt virus” nucleoprotein (H-CDR3-TSWV(BRO1)/6H, SEQ ID NO: 21 and L-CDR3-TSWV(BR01)/6H, SEQ ID NO: 22; H-CDR3-TSWV(P105)/1C, SEQ ID NO: 23 and L-CDR3-TSWV(P105)/1C, SEQ ID NO: 24), and

“cucumber mosaic virus” (H-CDR3-CMV/4G, SEQ ID NO: 25 and L-CDR3-CMV/4G, SEQ ID NO: 26; but also H-CDR3-CMV/4B, SEQ ID NO: 27 and L-CDR3-CMV/4B, SEQ ID NO: 28; and finally H-CDR3-CMV/2G, SEQ ID NO: 29 and L-CDR3-CMV/2G, SEQ ID NO: 30).

The term H-CDR3 in FIG. 7 indicates position of the peptides H-CDR3-LYS/11E, H-CDR3-BSA/9F, H-CDR3-TSWV(BRO1)/6H, H-CDR3-TSWV(P105)/1C, H-CDR3-CMV/4G, H-CDR3-CMV/4B in the VH region. The term L-CDR indicates position of peptides L-CDR3-LYS/11E, L-CDR3-BSA/9F, L-CDR3-TSWV(BRO1)/6H, L-CDR3-TSWV(Pl05)/1C, L-CDR3-CMV/4G and L-CDR3-CMV/4B in the VH region.

The L-CDR1-MUT peptide(SEQ ID NO:94) constitutes also an object of the present invention.

All the above CDRs peptides can be used for the detection of molecule of interest in a sample according to the techniques known in the art; said use shall be considered included in the object of the invention.

A further object of the present invention is given also by all the antibodies which include at least one of the above mentioned VH and VL polypeptides of the present invention.

In this regard, not only engineered antibodies of the scFv type, but also engineered antibodies of FAb, Fv, dAb type as well as all the immunoglobins, in particular those of the type IgG and IgA are object of the present invention.

In particular, object of the present invention are the engineered antibody scFv(F8) (SEQ ID NO: 49) and the scFv antibodies: scFv(P5) (SEQ ID NO: 50), scFv(LYS11E), scFv(BSA9F), scFv(BR01-6H), scFv(P105-1C), scFv(CMV-4G), scFv(CMV4B), and scFv(CMV-2G).

Such scFv antibodies are obtainable by connecting the respective VH and VL polypeptides with a linker covalently bound to the carboxy-terminal end of the VH polypeptide through its amino-terminal end, and to the amino-terminal end of the VL polypeptide through its carboxy-terminal extremity (see the diagram of in FIG. 2).

This linker can be one of any of the linkers known in the art to be suitable for connecting VH and VL polypeptides. In particular can be the linker with the sequence given as SEQ ID NO: 51 in the list of sequences in the annex.

All variants of the above mentioned peptides and polypeptides or the antibodies containing them must also be considered included in the object of the present invention.

These variants include:

a muteine of the above mentioned FRs and CDRs peptides, VH and VL polypeptides and antibodies including them,

a molecule including at least one of the above mentioned FRS and CDRs peptides, VH and VL polypeptides and antibodies including them, or at least one of the relevant muteins,

a fragment or a part of the above mentioned FRs and CDRs peptides, VH and VL polypeptides and antibodies including them, or at least one of the relevant muteines,

a fragments or a part of the molecules which include at least one of the above mentioned FRs and CDRs peptides, VH and VL polypeptides and antibodies including them, or at least one of the relevant muteines,

all as essentially consisting of at least one of the above mentioned FRs and CDRs peptides, VH and VL polypeptides and antibodies including them.

Each molecule derived directly or indirectly from one of the peptides, polypeptides and antibodies of the present invention and in particular the ones having the sequences reported in the annexed sequences listing must be considered as essentially consisting of one of said peptides, polypeptides and antibodies, when:

in the case of the variants of the above mentioned peptides/polypeptides, is contained in antibodies which are functional and stable at least in the cytoplasm of any cell;

in the case of variants of the above mentioned antibodies is itself an antibody which is functional and stable at least in a cytoplasmic compartment.

For the purposes of the present application, a muteine of the above mentioned peptides/polypeptides/antibodies similar to the original peptide/polypeptide/antibody so that it can suggest a derivation of the second from the first.

A fragment of at least one of the above mentioned peptide/polypeptide/antibody or of one of its muteines or of a larger molecule larger includes it, is given for the purposes of the present invention by a molecule in which one or more amino acids of the original peptide or muteine have been truncated.

In regard to the larger molecule, which contains at least one of these peptides/polypeptides/antibodies and/or muteines, the portion of the molecule different from these peptides and/or muteines can be partly or totally coincident with the sequence of scFv(F8).

All these molecules can be produced by means of a series of conventional techniques known in the art such as for example recombinant DNA but also constructions of synthetic polypeptides.

A further object of the present invention is given by polynucleotides (both polydeoxyribonucleotides and polyribonucleotides) coding for the peptides/polypeptides of the present invention or for their variants. In particular polynucleotides having the sequences reported as SEQ ID NO: 52 (H-FR1-F8), SEQ ID NO: 54 (H-FR2-F8), SEQ ID NO: 56 (H-FR3-F8), SEQ ID NO: 58 (H-FR4-FB), SEQ ID NO: 60 (L-FR1-F8), SEQ ID NO: 62 (L-FR2-F8), SEQ ID NO: 64 (L-FR3-F8), SEQ ID NO: 66 (L-FR4-F8), SEQ ID NO: 68 (H-CDR1-F8), SEQ ID NO: 70 (H-CDR2-F8), SEQ ID NO: 72 (H-CDR3-F8), SEQ ID NO: 74 (L-CDR1-F8), SEQ ID NO: 76 (L-CDR2-8), and SEQ ID NO: 78 (L-CDR3-F8), SEQ ID NO: 80 (VH-F8), and SEQ ID NO: 82 (VL-F8), SEQ ID NO: 95 (L-CDR1-MUT), are included in the object of the present invention.

Included in the present invention are also polynucleotides coding for the engineered antibodies obtained using the peptides/polypeptides of the present invention and in particular those with sequences given as SEQ ID NO: 84 (scFv(F8) and SEQ ID NO: 86 (scFv(P5)).

A further object of the present invention is given by the peptides/polypeptides, engineered antibodies and/or polynucleotides of the present invention, for use as a medicament and in particular for the preparation of pharmaceutical compositions suitable in gene therapy, in particular for the therapy of all the pathological states associated with an accumulation of at least one molecule in an intra or extra cellular environment. Here in particular, reference is made to infectious, tumor, metabolic and immunitary (especially auto-immunitary) pathologies. In regard to the infectious pathologies, reference is made in particular to those associated with viruses, either in animals (see for example pathologies associated with the HIV, in particular HIV-1, HPV, Herpes Virus or HCV virus in humans), or in plants (see for example the pathologies associated with the CMV or TSWV viruses in tomatoes).

Further also the pharmaceutical compositions which include a therapeutically effective amount of at least one of the above mentioned peptides, polypeptide, antibodies and/or variant thereof, and/or a therapeutically effective amount of at least one of the above mentioned polynucleotides and a pharmaceutically suitable vehicle.

This pharmaceutically acceptable vehicle carrier or auxiliary agent can be any vehicle carrier or auxiliary agent known in the art as being suitable for preparing pharmaceutical compositions or material containing the above mentioned molecules. In particular according to the invention such a carrier can be the liquid or solid carrier which are used or in any case known by a person skilled in the art to be suitable with protein and antibody.

Object of the present invention is also a method for the treatment of pathologies associated to the accumulation of a molecule inside or outside human, animal cell, comprising the step of

administering a therapeutically effective amount of above mentioned antibody or variant thereof to a subject in need thereof.

In particular said administration can be carried out parenterally for treating cancer and pathologies associated thereof, or by oral route as passive immunotherapy, according administration techniques known to a person skilled in the art.

Object of the present invention is also a method for the treatment of pathologies associated to the accumulation of a molecule inside or outside human, animal or plant cell, comprising the step of

administering a therapeutically effective amount of above mentioned polynucleotides or variant thereof coding for the antibodies of the present invention to a subject in need thereof.

Said administration shall be carried out by inserting said polynucleotides in vectors suitable for trasfection. According to the present invention any of the vectors, in particular the viral ones, currently adopted by a person skilled in the art for treating subject in need thereof, can be used to this purpose.

A further object of the invention is the use of the above mentioned antibodies and/or polynucleotides or variants thereof for the treatment of pathologies associated to accumulation of a molecule inside or outside the human, animal or plant cell.

The use of these above mentioned peptide/polypeptide/antibody or a variant thereof for diagnosis of these pathologies is also included in the object of the present invention.

In particular such molecules can be used in diagnostic applications fused to genes encoding for protein having an enzymatic activity (i.e., alkaline phosphatase) or reporter proteins such green fluorescent protein. Other reporter genes or proteins known in the art as suitable in diagnostic application can be used as well.

Among the diagnostic application wherein the antibodies of the present invention can be used imaging is particularly preferred.

Object of the present invention is a diagnostic kit including the above molecules as reagents, and in particular a diagnostic kit for infectious pathologies characterized in that it includes at least one composition including at least one of the above mentioned polynucleotide, peptide, polypeptide, antibody and/or a variant thereof.

Included in the object of present invention is also the use of the above mentioned antibodies for identifying new molecules and/or the relevant function.

Such identification can be carried out for instance according to techniques known in the art referred to genomics and proteomics.

In particular object of the present invention is the use of the above mentioned peptide/polypeptide/antibody or a variant thereof, for deriving molecules to be used in therapy of infectious pathology, and the relevant kit including—at least one of such peptide/polypeptide/antibody or a variant thereof as a reagent.

Further object of the present invention is also a composition suitable in experimental applications including at least one of the above mentioned molecules and a vehicle chemically compatible with them.

This chemically compatible vehicle can be any vehicle known in the art as being suitable for pharmaceutical compositions or material containing the above mentioned molecules.

In particular according to the invention such a carrier can be the liquid or solid carrier which are used or however known by a person skilled in the art to be suitable with protein and antibody.

The invention will be better described with the help of the figures in the annex.

### DESCRIPTION OF THE FIGURES

FIG. 1 shows the arrangement of the peptides of the present invention in the VH and VL polypeptides of an antibody according to the present invention.

FIG. 2 shows the connection of the VH and VL polypeptides of the present invention in antibodies of the scFv type.

FIG. 3 shows the summary diagram of the modifications of the sequence of scFv(F8) which do not alter the actual characteristics of stability of the antibody. The regions (FRs and CDRs) are individualized according to AbM (Oxford Molecular Ltd) and the numbering is according to Kabat (Kabat et al 1991).

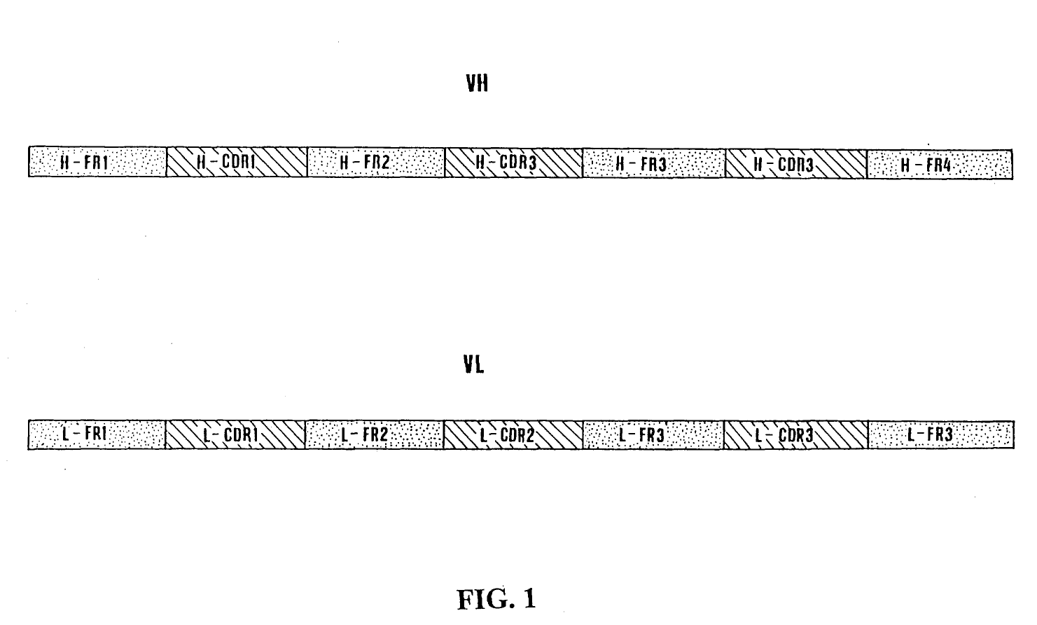

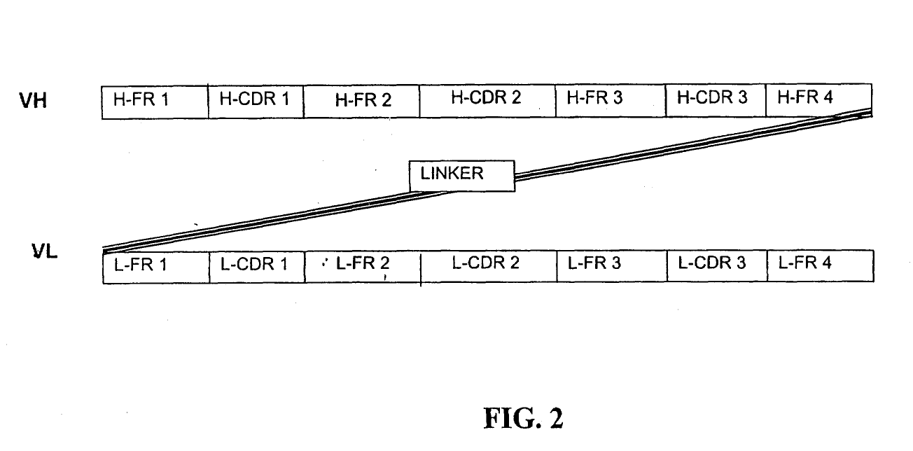

The residues shown in gray colored squares are the residues which must remain unchanged, the residues shown in black colored squares are the residues which can be substituted with any amino acid, the residues shown in white squares are modifiable as follows:

the residue VH 24 is substitutable with residues of equivalent chemical nature;

the residue VH 47 is substitutable with residues of equivalent chemical nature;

the residue VH 52 is substitutable with residues of equivalent chemical nature or different chemical nature, or can be eliminated by deletion;

the residue VH 60 is substitutable with residues of equivalent chemical nature or different chemical nature;

the residue VH 61 is substitutable with residues of equivalent chemical nature or different chemical nature;

the residue VH 71 is substitutable with residues of equivalent chemical nature;

the residue VH 76 is substitutable with residues of equivalent chemical nature;

the residue VH 78 is substitutable with residues of equivalent chemical nature;

the residues VH 100-10OG are substitutable with residues of equivalent chemical nature or different chemical nature, or can be eliminated by deletion;

the residues VL 27C-29 are substitutable with residues of equivalent chemical nature or different chemical nature, or can be eliminated by deletion;

the residue VL 68 is substitutable with residues of equivalent chemical nature or different chemical nature;

the residue VL 71 is substitutable with residues of equivalent chemical nature or different chemical nature.

FIG. 4 shows a summary diagram of the strategy of mutagenesis adopted by means of rational substitutions of the VH and VL polypeptides of the antibody scFv(F8). The actual amino acidic residues of the sequence of scFv(F8) are shown in normal characters, the amino acids of the sequence of scFv(Dl.3) (Bhat et al. 1990) are shown in underlined characters. The sequences are shown for all the products of intermediate mutagenesis (P1, P2, P3 and P4) and for the last product of mutagenesis (P5), so that the modifications (substitutions and deletions) effected on the original sequence of scFv(F8) are marked by underlining.

FIG. 5 shows the modification effected on the CDR1 of the VL according to the approach of casual mutations for the derivation of mutants of scFv(F8).

FIG. 6 shows the oligonucleotides used as primers for the construction of the “monoscaffold” library as described in the examples. The term N indicates any nucleotide, the term M indicates the DATP or dCTP nucleotide.

FIG. 7 shows the layout of the peptides of the present invention in the VH and VL polypeptides of antibodies in which the binding specificity is in particular given by the H-CDR3 and L-CDR3 peptides.

### DETAILED DESCRIPTION OF THE INVENTION

As mentioned above the peptides of the present invention are capable of rendering the antibodies including them stable even in a reducing environment.

Regions determining the complementarity (CDRs) with the antigen and framework regions (FRs) of scFv(F8) antibody have been identified by sequencing according to criteria known in the state of art.

ScFv(F8) has been used as a scaffold onto which new specificities have been engineered either by loop grafting (“rational approach”) or by mutation and selection through repertoire generation (“molecular evolution”) Antibodies deriving from site-directed mutagenesis have been analyzed to verify maintenance of stability and solubility in cytoplasmic environment and the acquirement of binding specificity different from AMCV.

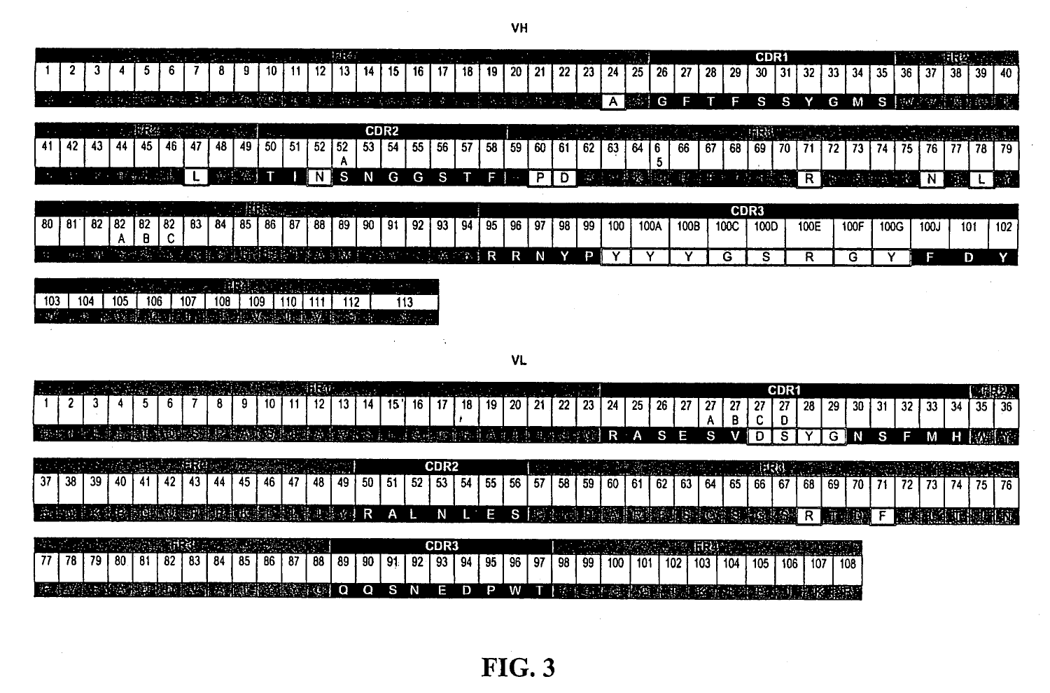

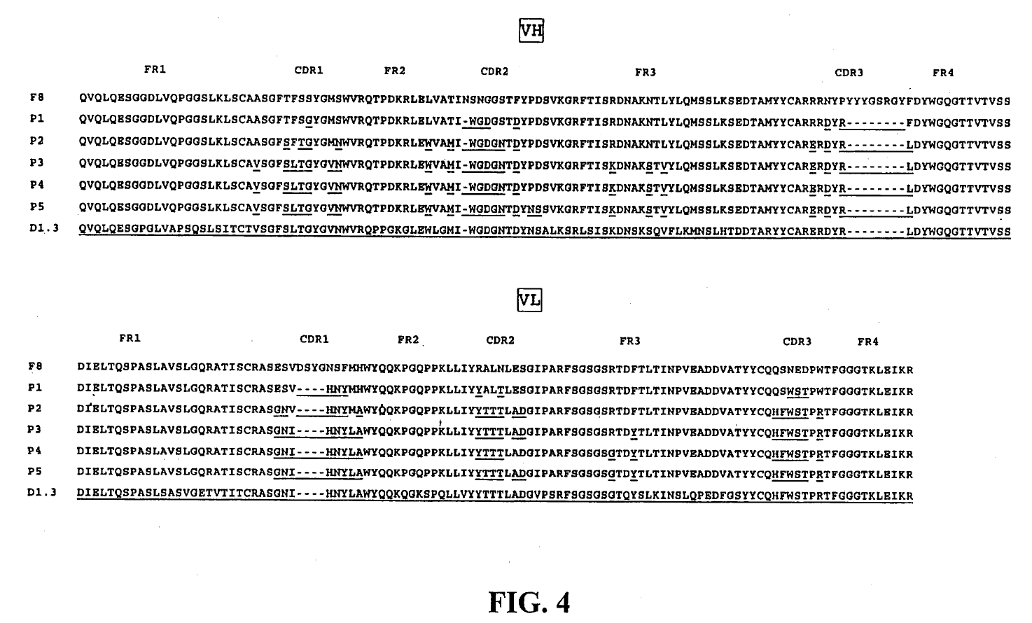

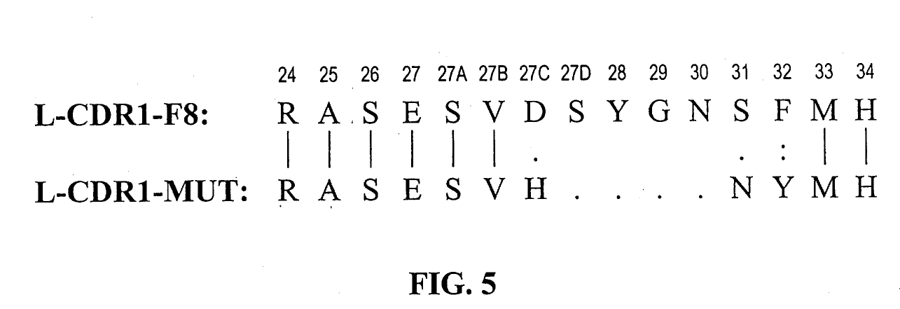

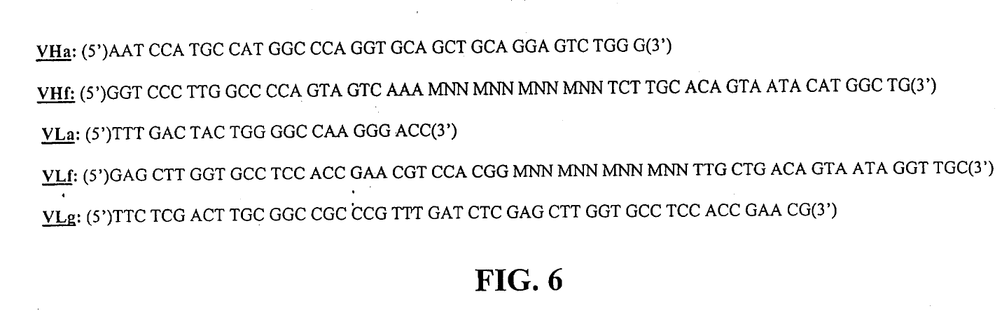

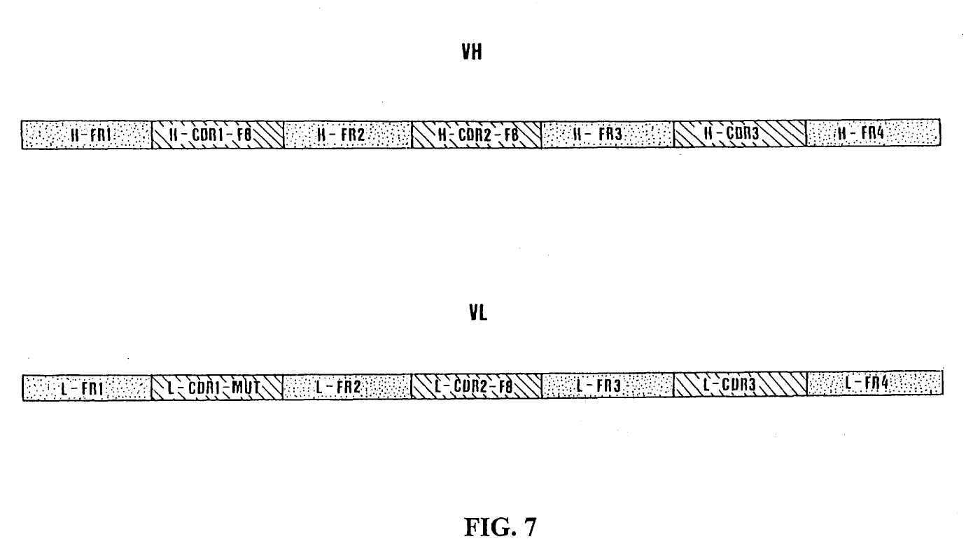

In the “rational approach” residues of both CDRs and FRs regions were substituted with residues of scFv(Dl.3) that recognizes lysozyme, whereas in “molecular evolution” residues of the CDR3 regions were casually substituted and the binding properties of the resulting scFv were checked. These experiments demonstrated that scFv(F8) scaffold tolerates substitution of defined residues in both CDRs and FRs regions, which can be redefined in terms of stability preservation of the antibody scFv(F8) (see in particular FIG. 3).

“Rational substitution” Approach

The inventors substituted scFv(F8) aminoacids with aminoacids of the CDRs and FRs of scFv(Dl.3) which recognizes lysozyme. These substitutions were performed in a rational way, modifying each residue on the basis of a theoretical design formulated by means of molecular “modelling”.

According to this approach extensive aminoacid modifications have been made, including all CDRs of VH and VL. These regions were substituted (by means the technique known in art as “grafting”) with the corresponding regions of another antibody of “normal type” (which does not show particular stability or cytoplasmic functionality), which binds another target molecule which can be lysozime, bovine seroalbumin, nucleoprotein of tomato spotted wilt virus cucumber mosaic virus, or any other target molecule of interest.

The diagram shown in FIG. 4 summaries the strategy of mutagenesis adopted for scFv(F8) antibody grafting.

The analysis of mutagenised products, in the specific case shown in the figure referred to mutagenesis direct to have an antibody binding lysozime, demonstrated the success of grafting strategy. In fact, the resulting chimerical antibodies (named P2, P3, P4 and P5) were able to specifically recognise lysozime, as predicted by the molecular design. At the same time the capacity of scFv(F8) scaffold to support extensive mutations was demonstrated, particularly at the level of the CDRs, without affecting molecular stability. In fact, the new binding molecules obtained with this approach showed to be-soluble and functional in the reducing environment of cell cytoplasm as well as the cognate scFv(F8).

As a result, except for few FRs residues modified to allow the correct folding of the regions involved in the new antigen recognition, the support structure of scFv(F8) was not substantially altered. The structure capable of functioning as “central nucleus” was identified, on which it is possible to graft, by means of protein engineering, sequences capable to confer binding ability for new antigens. This central nucleus is composed of the FRs peptides described in the summary of the invention.

The properties of these mutant molecules were verified by the inventors by means of techniques of analysis of the expression in both periplasm and cytoplasm of Escherichia coli, better explained and exemplified in the examples. In particular, the analysis of the expression in the bacterial periplasm has enabled to evaluate the ability of the grafted antibodies to recognise the new antigen. The expression in the bacterial cytoplasm has instead permitted to analyze protein solubility and functionality in reducing environment.

In the case of antibodies mutagenized for lysozime specificity the ELISA (Enzyme Linked Immunosorbent Assay) test carried out on periplasmic extracts normalized for total protein content has shown that all the products of mutagenesis, with the exception of P1 (the mutant with the least number of substitutions), were able to recognise lysozime. The P3 mutant, which presents the CDRs of scFv(Dl.3) and some substitutions in the frameworks, showed the highest binding activity. Lysozime recognition observed for P3 was slightly lower than those showed by scFv(Dl.3) (about 15% less). These data were combined with the Western blot analysis which showed comparable expression levels of the various products of mutagenesis and between mutagenesis products and the original scFv(F8) and scFv(Dl.3). This indicate that the differences of signal observed in ELISA are exclusively due to different specificity for the antigen, rather than to different expression levels.

In regard to the expression in the bacterial cytoplasm, ELISA carried out on extracts normalized for total proteins gave a higher activity for P5 mutant, characterized by the highest number of aminoacidic substitutions. A poor binding activity, in each case higher than that measured for scFv(Dl.3), was also registered for P3 and P4.

Even in this case Western blot analyses showed an equivalent expression of the scFv mutants expressed in the cytoplasm, confirming that, once again, the differences of ELISA signal are attributable to the different ability of recognition of the lysozyme.

“Molecular evolution” Approach

On the basis of the indications obtained from the rational substitution approach, a library of new molecules, derived from scFv(F8) has been construed.

The starting point for library construction was scFv(MUT-VL1), a derivative of scFv(F8) antibody, which was modified in the CDRs. The CDR1 of the VL domain was shortened and partially modified according to a modeling-assisted design. Moreover, 9 aminoacids were removed from the unusually long CDR3 of the VH domain of the original scFv (see FIG. 5).

Structural variability was introduced by targeted random PCR-mutagenesis in four aminoacid positions in the CDR3 of both VH and VH domains. Degenerated oligonucleotides, randomizing residues between and including 95 and 98 of VH and 91 and 94 of VL, were used. After mutagenesis, a repertoire with an estimated diversity of 5×10⁷ different phage clones was obtained.

The pool of molecules thus obtained was mounted on phage in a library for the selection on antigens different from AMCV, originally recognized by scFv(F8). This “library”, constructed—using as a basic structure a single starting antibody (“monoscaffold library”), provided antibodies with different specificity, which at the same time retain the peculiar characteristics of stability of scFv(F8) molecule (cytoplasmic solubility, denaturation and renaturation in vitro). These conclusions were arrived at by guanidinium chloride denaturation/renaturation studies of isolated scFv molecules. The expression of the same ScFv fragments in the Escherichia coli cytoplasm as soluble and functional molecules confirmed the stability of these proteins also in in vivo system.

Further, for some of them (scFv(CMV)4B and scFv(CMV) 4G), it has been shown that they are expressed as soluble and functional molecules also in the plant cytosol. The presence of scFv(CMV)4B or scFv(CMV)4G antibodies in the soluble fraction of potato virus X (PVX)-infected tobacco plants shows that these molecules, differently from most antibodies, are folded and stable in the plant cytoplasm. Identical results were achieved in transgenic tomato plants, confirming the capability of a correct folding in a reducing environment. These antibodies interfere with CMv assembly and/or replication, providing an engineered resistance to the virus (‘plantibody-mediated resistance’).

CDR3 sequences of VH and VL domains of the antibodies isolated from the library and characterized (see table I infra) showed that residues substitution in the above mentioned positions is totally casual. Moreover, aminoacid composition and distribution in mutated CDR3 indicated that charge and hindrance of aminoacids do not substantially affect the folding and solubility of these scFv fragments.

The data obtained from the construction and from the analysis of the “monoscaffold library” has shown the possibility of using the scaffold of scFv(F8) to construct a pool of polyvalent antibodies with the same intrinsic stability as the original antibody scFv(F8). This library is destined to all applications in which the expression of antibodies in the cytoplasm is required or where particular characteristics of molecular stability are essential.

As a result of these experiments, the stable antibody molecules with a new binding specificity and in particular the ones described in the summary of the invention have been obtained.

Conclusions

The results of all these experimental approaches are summarized in the diagram shown in the following table I.

In this table the results and the information obtained as a result of the derivation of mutants through the approach of casual substitutions are reported. The non polar residues are shown with double underlined characters, the residues with polar R group with no charge are shown in normal characters; the acid residues (negative charge) are shown with dashed underlining and the basic residues (positive charge) are shown with single underlining.

TABLE I

| Clone | Specificity | VH CDR3^{a} | VL CDR3^{b} |
|---|---|---|---|
| scFv(F8) | AMCV | RRNYPYYYGSRGY | [symbol] |
| scFv(LYS-11E) | Lisozyme | [symbol] | VTYK |
| scFv(BSA-9F) | BSA | [symbol] | ALSP |
| scFv(BR01-6H) | Nucl. of TSWV | [symbol] | GASI |
| | from BR01 | | |
| scFv(P105-1C) | Nucl. of TSWV | GRHK | YGRR |
| | fromP105 | | |
| scFv(CMV-4G) | CMV | NNWS | GQRK |
| scFv(CMV-4B) | CMV | NNYS | [symbol] |
| scFv(CMV-2G) | CMV | VTYN | [symbol] |

^{a}indicates: when referred to scFv(F8), the positions from residue 95 to residue 100J; when referred to clones isolated from “monoscaffold library” the positions from residue 95 to residue 98 (numeration according to Kabat et al., 1991).
^{b}indicates positions from residue 91 to residue 94 (numeration according to Kabat et al., 1991).

On the basis of the above results the peptides, polypeptides, antibodies reported in the summary of the invention have been obtained following the process reported also therein.

The antibodies obtained thereby in particular have been proved for the improved stability, and in the antibodies of the type FAB, Fv, dAb, IgG or IgA have in particular shown an improved stability due to the presence of the peptides of the invention in the VH and VL regions.

Up until now a general description of the present invention has been given. With the help of the following examples, a more detailed description will now be provided, in order to clarify scope, characteristics, advantages and operating method.

### EXAMPLES

### Example 1

### Determination of the Sequence of scFv(F8)

The single chain antibody scFv(F8) derives from a MAb (secretion from a hybridoma) of the class IgG2b directed against the coat protein of the plant virus AMCV (artichoke mottle crinkle virus). To obtain this antibody, Balb/c mice were immunized with the purified virus. The techniques for isolating the lymphocytes and obtaining the hybridoma were the standard ones reported in literature (Harlow and Lane 1988). The hybridoma line expressing this antibody was selected by ELISA, on the basis of its high affinity for the antigen. To isolate the genes of the heavy (H) and light (L) chain of this antibody, the complete cDNA were selected according to published protocol (Tavladoraki et al. 1993). The variable regions (VH and VL) were amplified by means of PCR (polymerase chain reaction) using universal primers for the variable regions and successively cloned in different types of vectors (Tavladoraki et al. 1993). The sequence was determined according to the Sanger method (Sambrook et al 1989) following the protocol of the Sequenase kit (USB).

### Example 2

### Determination of the Site Specific Mutagenesis in the Rational Approach

The mutagenesis was carried out on the plasmid pMUT-VL1, containing the gene that codes for an antibody derived from the scFv(F8), from which only the CDR1 of the VL is differentiated for, partially modified in the aminoacidic composition and reduced by four amino acids.

The Stratagene (“Quick Change Site-directed Mutagenesis Kit”) system of mutagenesis was used following the indications provided by the manufacturer. This system is based on the enzymatic extension of two complementary primers containing mutated sequences, using pMUT-VL1 as a template. The plasmid was denatured allowing the annealing of the two oligonucleotides to the opposite DNA strands, so as to prime the extension reaction of the Pfu DNA polymerase. The result is a plasmid with a double helix that has incorporated the oligonucleotides causing the desired mutation.

The bacterial stock of E. Coli XL1-Blue used for the purification of the plasmidic vector has methyl-transferase activity, therefore the DNA plasmid extracted from bacteria results methylated. On the contrary the mutant plasmids obtained by extension of the Pfu DNA polymerase, do not contain methylations. This has enabled the selection of mutated plasmids by digestion of the products of reaction with the endonucleasic enzyme DpnI that recognizes frequent specific sequences of methylated DNA in the parent plasmid. The products of the reaction were transfected in E. Coli XL1-Blue where the “nick” sites, produced during the synthesis in vitro of the mutant plasmid are phosphorylated by the cellular repair systems.

The mutagenesis reaction was carried out in 25 μl containing: 2.5 μl reaction buffer 10×, 2.5 mM dNTP, 10 ng DNA template, 62,5 ng each primer, 1.25 U Pfu DNA polymerase (Stratagene).

The conditions adopted were the following: denaturation at 95° C. of 30″; 18 cycles: denaturation at 95° C. for 30″, annealing at 55° C. for 1′ and enzymatic extension at 68° C. for 7′; 5′ at 68° C. to complete the extension. The reaction was performed using a Perkin Elmer/Cetus apparatus.

The products of the reaction were incubated for one hour at 37° C. with 5 U of the restriction enzyme DpnI (Stratagene) and analysed for electrophoresis on agarose gel.

The products of mutagenesis were cloned in pGEM-7Zf(+) vector (Promega) and transfected in E. Coli XL1-Blue, according to the conventional methods of molecular biology. Mutagenesis was verified by sequencing.

Proteins expressed by mutated sequences were analysed after cloning in the expression vector pDN332 (Neri et al. 1996) and induction of expression in bacteria, according to the technique described in the following examples.

The functionality of the antibodies and the solubility in cytoplasmic reducing environment were analysed by means of Western blotting and ELISA, as described below.

### Example 3

### Extraction of the Protein Expressed in E. Coli Periplasm

Single colonies of E. Coli HB2151 expressing scFv antibodies were inoculated in 50 ml SB culture medium containing 100 μg/ml ampicillin and 2% glucose and grown for 16 hours at 37° C. This preculture was diluted up to 1 litre with fresh SB medium and underwent shaking at 37° C. for two additional hours (until A_{600 nm}=0.7−0.8) Then cells were centrifuged at room temperature at 3000 g for 15′.

The induction of the lacZ promoter was obtained by resuspending the bacterial precipitate in 1 litre SB medium containing 100 μg/ml ampicillin and 1 mM IPTG and shaking at 30° C. for 3 hours. The culture was then centrifuged at 3000 g for 15′ at 4° C. to precipitate the cells.

Soluble proteins were extracted from bacterial periplasm by osmotic “shock”. The pelleted cells were resuspended in 15 ml of TES buffer solution (0.5 M saccarose, 0.2 M Tris-HCl, 0.5 mM EDTA, pH 8.0) containing protease inhibitors (“Complete Mini”, Boehringer).

Then 22.5 ml of 1:5 diluted TES and protease inhibitors were added. The bacterial suspension was subjected to a slow rotation for 15′ at room temperature. The extract was then centrifuged at 15000 g, at 4° C. for 20′ to recover periplasmic proteins present in the supernatant.

### Example 4

### Expression of Proteins in E. Coli Cytoplasm

a. Preparation of Intracellular Constructs

To obtain the expression of scFv fragments in the bacterial cytoplasm signal sequenceless constructs were prepared. The sequence, encoding the scFv(F8) devoid of the PelB secretion signal (Tavladoraki et al., 1993), was cloned in HindIII-NotI sites of pDN332 phagemid, yielding the phagemid named pDN-F8intra. Constructs of scFv genes destined to intracellular expression were obtained by substituting a PstI-NotI 744 bp fragment of pDN-F8intra, corresponding to the scFv(F8) gene, with the analogue PstI-NotI restriction fragments obtained by digestion of mutated scFv genes. Plasmids containing the signal sequenceless version of the scFv genes were used to transform E. coli HB2151.

b. Expression of Recombinant Protein in E. Coli

Fifty ml of 2xTY-AG were inoculated with an overnight culture at A_{600 nm}=0.05. Cells were grown at 37° C. until A_{600 nm}=0.6, then they were pelleted and resuspended in 50 ml SB medium containing 100 μg/ml ampicillin and 0.4 mM IPTG. After 3 h induction at 30° C., bacteria were collected in 2.5 ml extraction buffer (20 mM Tris-HCl, 2 mM EDTA, 10 mM iodacetamide, pH 8), frozen/thawed at −20° C., and then sonicated for 3 min at 150 Watt power (Soniprep 150, Sonyo) on ice. The soluble fraction was collected by centrifugation for 30 min at 18000 g.

The pellet, containing inclusion bodies, was resuspended in electrophoresis buffer (20% glycerol, 2% SDS, 0.06 M Tris-HCl pH 6.8, 0.02% bromophenol blue, 5% β-mercaptoethanol), boiled for 5′ and the supernatant obtained after a brief centrifugation was loaded on polyacrylamide gel.

### Example 5

### Analysis of Recombinant Antibodies

a. Determination of the Protein Concentration

The concentration of the total protein present in the extracts was determined using the BioRad reagent and bovine seroalbumin as a standard.

The concentration of the scFv after purification was calculated on the basis of absorbance at 280 nm. After electrophoretic separation protein bands were visualised with silver nitrate or Coomassie blue staining methods.

b. Electrophoresis on Polyacrylamide Denaturing Gel (SDS-PAGE)

The analysis of the antibody fragments obtained after purification was carried out by means of SDS-PAGE according to the protocol known in art. The separation gel was prepared using polyacrylamide (acryilamide/bi-acrylamide 29:1) at 12% final concentration, in the presence of 2% SDS. Loading buffer (final concentrations: 10% glycerol, 0.06 M Tris-HCl pH 6.8, 0.025% bromophenol blue, 2% SDS and 5% β-mercaptoethanol) was added to the samples before boiling for 5′. Electrophoresis was carried out using MiniProtean (BioRad) apparatus at 100 Volt.

c. Western Blot Analysis

After separation on SDS-PAGE, the proteins were then electro-transferred from the gel onto a nitrocellulose membrane (Hybond-C Super, Amersham) at 100 Volt for one hour at 4° C.

The nitrocellulose membrane was then blocked in PBS buffer (0.2 M NaH₂PO₄, 0.2 M Na₂HPO₄, 0.15 M NaCl) containing 0.1% Tween-20 (PBST) and 4% skimmed milk, at 4° C. for 16 hours.

After three washes with PBST, respectively of 30″, 5′, 5′, and a wash of 5′ in PBS, the membrane was incubated in PBS, 2% skimmed milk (PBSM) containing 2.5 μg/ml monoclonal antibody anti-Flag M2 (Sigma), for 2 hours at room temperature. The membrane was washed and immunodetection was realised by incubation for 1 hour at room temperature in PBSM containing biotinylated anti-mouse IgG antibody (Amersham), followed by incubation in PBSM with streptavidin-horseradish peroxidase conjugate (Amersham). Signal development was obtained by enhanced chemioluminescence (ECL Plus, Amersham).

d. ELISA Test

Immunoplates (Maxisorp, Nunc) were coated with antigens (100 μg/ml lysozyme (Sigma) and 3 μg/ml AMCV) in 100 μl carbonate buffer (50 mM NaHCO₃ pH 9.6), at 4° C. for 16 hours.

After 3 washes with PBST and 1 wash with PBS, the plates were blocked with PBSM for 2 hours at 37° C. Each well was then washed and filled with 80 μl extract from induced bacterial cultures and 20 μl PBSM containing 2.5 μg/ml anti-FlagM2 (Sigma). The plates were incubated for 16 hours at 4° C. and washed. 100 μl PBSM were then added to each well containing a goat anti-mouse antibody conjugated to the peroxydase (KPL). The plates were incubated at 37° C. for one hour and washed. For signal development 100 μl of 1:1 solution of peroxydase substrate, ABTS [acid 2′2′-azinebi(3-ethylbenzthiazolin) sulphonic]: H₂O₂ (KPL) were used. Signals were measured at 405 nm absorbance by means of ELISA reader (Labsystem Multiscan Plus).

### Example 6

### Construction and Cloning of the “Monoscaffold” Library

As a template for the construction of the phage library the plasmid pMUT-VL1 described in example 2 was used. The library was obtained by introducing variability in the CDR3 of the VH and VL, through the random modification of the sequence encoding four aminoacidic residues in the positions indicated in table I. For this purpose, partially degenerated oligonucleotides were synthesized, designed to avoid the introduction of transcription stop codons and to reduce the length of the CDR3 of the VH from 13 to 4 aminoacids. The mutagenesis was carried out by means of PCR, independently amplifying the coding sequences for the VH and VL domains, using respectively, the primers VHa and VHf and VLa and VLf (FIG. 6). The reaction of PCR was prepared as follows: 300 ng pMUT-VL1, 0.4 mM sense primer (VHa or VLa), 0.8 μM degenerated antisense primer (VHf or VLf), 250 μM of each DNTP, in 50 μl of incubation buffer 1× Appligene, (Oncor); 2.5 U of Taq DNA Appligene polymerase, (Oncor) were added at 94° C. (hot start). The amplification reaction was performed according to the following program: 94° C. for 3 minutes; 25 cycles: 94° C. for 1 minute, 60° C. for 1 minute, 72° C. for 1 minute; 72° C. for 2 minutes. Amplification products were purified from agarose gel, using the QIAquick Gel Extraction Kit (QIAgen), eluting in 30 ml of 3 mM Tris/HCl pH 8.0. The assembly of the scFv antibodies was carried out by means of PCR under the same conditions as described above, using 10 μg of the purified VH and VL fragments, 0.8 μM sense primer (VHa) and 0.8 μM antisense primer (VLg), in a final volume of 500 μl subdivided into 10 reactions of 50 μl. The assembled scFvs were purified using the QIAquick PCR Purification Kit (QIAgen). After NcoI-NotI digestion, the entire product of assembly was cloned in the vector pDN332.

Ligation products were transfected by electroporation in the E. Coli TG1 strain, adopting the following conditions: 200 ohm, 25 mF, 2.5 kvolt. The transformants were selected on 2xYT +2% glucose +ampicillin 100 g/ml. The phages were prepared according to the protocol described by Nissim et al. (1994).

### Example 7

### Selection of the Mutants from the “Monoscaffold” Library and Analysis of Their Properties

a. Selection of the Library

Antigens immobilization on immunotubes (Maxisorp, NUNC) was carried out in PBS (8 mM Na2HPo₄.12H₂O+1 mM NaH₂PO₄.H₂O+0.15M NaCl) or in carbonate buffer 50 mM (CB), at concentration varying between 10 and 100 μg/ml depending on the antigen, incubating for 16 hours at room temperature or at 4° C.

10¹²-10¹³ phages in 4 ml of PBS +2% milk (PBS-M) were used for each selection cycle, incubating for 2 hours, with the first 30 minutes of slight agitation. After 10-15 washes with PBS +0.1% Tween20 and 10-15 washes with PBS, the elution was carried out with 1 ml of 100 mM triethylamine, immediately neutralized with 0.5 ml of 1 M Tris/HCl pH 8.0. The phage suspension was then used to infect 10 ml of E. Coli TG1 strain (37° C. for 30 minutes) exponentially growing. After centrifugation, the bacteria were resuspended and plated on plates containing agar medium: 2xYT +100 μg/ml ampicillin +1% glucose (2xYT-AG).

The characterization for binding ability of single clones deriving from the last panning cycles was carried out in some cases on clones expressed in the form of phage and in others expressed as soluble scFv.

In the case of phage clones, 96 single colonies obtained from the last selection cycle were inoculated in 150 ml of 2xYT-AG and grown at 30° C. in agitation for 16 hours. Then an aliquot of 10 ml of each pre-culture was used to inoculate 150 ml of 2xYT-AG until reaching exponential growth. The production of phages was obtained by infecting with about 10¹¹ t.u. (transforming units) of ‘helper’ phage VCSM13 (Stratagene) and incubating at 37° C. for 30 minutes. The infected bacteria were centrifuged, resuspended in 150 ml of 2xYT +100 μg/ml ampicillin +25 μg/ml kanamicine and incubated for 16 hours at 30° C. The culture supernatants were analyzed by ELISA, with antigens immobilized under the same conditions used for the immunotubes. After incubating 2 hr at 37° C., a peroxidase-conjugated monoclonal antibody anti-M13 (Pharmacia) was used 1:5000 in PBS-M, 1 hour at 37° C. The colorimetric reaction was developed using the “ABTS peroxidase substrate system” (KPL). Positive clones obtained from this preliminary selection were further analyzed in order to verify the functionality as soluble scFvs. For this purpose, plasmids from positive clones were extracted by means of the QIAprep Spin Miniprep (QIAGEN), sequenced and transfected in the E. Coli HB2151 strain. Competent bacteria were made by resuspension at 0° C. in TSS (for 100 ml: 1 gr bacto-triptone, 0.5 gr yeast extract, 0.5 gr NaCl, 10 gr PEG 3350, 5 ml DMSO, 50 mM MgCl₂, pH 6.5). DNA plasmid (1-5 ng) was added to-1 ml of competent cells and incubated on ice for 45 minutes. After a brief shock at 42° C. for 2 minutes, 1 ml of LB was added, then the cells were placed in agitation at 37° C. for 1 hour and plated on LB+100 μg/l ampicillin. The analysis of expression of single colonies from transformation was carried out by ELISA, according to the method described below.

For selection as soluble scFvs, 10-100 μl of phages, eluted from the last panning on antigens, were used to infect cells of the E. Coli HB2151 strain in exponential growth. 96 single colonies, were inoculated 150 μl of 2xYT-AG for 16 hours in agitation. 10 μl of each pre-culture were diluted in 150 μl of 2xYT +100 μg/ml ampicillin +0.1% glucose and grown at 37° C. for 1 hour. Protein expression was induced by adding 1 mM IPTG, 16 hours incubation. Single culture supernatants (containing 2.5 μg/ml of an anti-FLAG M2 monoclonal antibody, Sigma, in PBS-M) were incubated 2 hr at 37° C. and analyzed by ELISA. A rabbit anti-mouse antibody (KPL), conjugated to the peroxydase, diluted 1:10000 in PBS-M wa then added 1 hour at 37° C.

b. Analysis of Cross-Reactivity

Positive clones derived from ELISA screening were further analysed for their binding specificity on antigens other than those selected on. Fifty ml of E. Coli HB2151 strain in exponential growth in SB-A (35 g/l tripton +20 g/l yeast extract +5 g/l sodium chloride+100 μg/ml ampicillin, pH 7.5) were induced at 30° C. for 3 hours by additing 1 mM IPTG. After centrifugation, the cells were resuspended in 500 μl of TES (0.2 M Tris-HCl pH 8+0.5 mM EDTA +0.5 M saccarose) containing protease inhibitors (Complete™, EDTA-free, Boehringer), then 750 μl of TES diluted 1:5 were added and the sample was incubated in orbital agitation for 10 minutes at room temperature. Protein extracts, obtained as supernatant after centrifugation for 20 minutes at 18000 g at 4° C., were used in an ELISA test with different antigens. In each ELISA well 80 μl of periplasmic extract were loaded then 20 l PBS +10% milk. Detection was carried out by using the antibody anti-FLAG M2, as described.

c. Sequencing

Plasmids from positive selected clones were sequenced on both DNA strands by using a 373 DNA Sequencer (Applied Biosystems).

d. Purification of the scFv

In order to produce high amounts of scFv, a single colony of E. Coli HB2151 was inoculated in 100 ml of SB containing 100 μg/ml ampicillin and 2% glucose (SB-AG). After 16 hours incubation at 30° C., the culture was diluted with 900 ml of SB-AG and left in agitation for a further hour (up to O.D.₆₀₀=0.9). After centrifugation, pellets were resuspended in SB-A+1 mM IPTG and induced for 3 hours at 30° C. After centrifugation, the bacteria were resuspended in 10 ml of TES +protease inhibitors, then 15 ml of TES diluted 1:5+protease inhibitors were added and the sample was incubated for 10 minutes at room temperature. After centrifugation (20 minutes at 18000 g at 4° C.), the supernatant was recovered (fraction 1A) and the pellet was resuspended in 15 ml of 5 mM MgSO₄+protease inhibitors for a further protein extraction. After 10 minutes incubation at room temperature, a second centrifugation step was performed and the supernatant was kept separately as fraction 2A. The fractions 1A and 2A were concentrated independently by ultrafiltration on Diaflo YM10 membrane (Amicon) and purified by chromatography of affinity on protein-L Sepharose (Actigene) or on Ni-NTA (QIAgen), according to the protocols suggested by the manufacturers. Quantification was carried out by reading the absorbance at 280 nm and protein purity was verified on SDS-PAGE followed by AgNO₃ staining.

e. Analysis of Thermodynamic Stability

i) Unfolding and refolding studies of scFv mutants.

Equilibrium unfolding experiments were performed at 20° C. by incubating each scFv mutant (35 μg/ml) at increasing guanidinium chloride (GdmCl) concentrations (0-4 M) in PBS. After 3 h, intrinsic fluorescence emission spectra were recorded at 20° C. To test the reversibility of the unfolding, the mutants (0.70 mg/ml) were unfolded at 20° C. in 4 M GdmCl in PBS for 3 h. Refolding was started by 20-fold dilution at 20° C. into solutions of the same buffer used for unfolding containing decreasing GdmCl concentrations. After 3 h, intrinsic fluorescence emission spectra were recorded at 20° C. Equilibrium unfolding experiments under reducing conditions were performed by incubating for 3 h at 20° C. 35 μg/ml of scFv(HEL-11E) in 20 mM Tris-HCl pH 9.0, 0.15 M NaCl, 2 mM DTT and 0.1 mM EDTA at increasing GdmCl concentrations (0-4 M) before recording fluorescence emission spectra. For refolding experiments, the mutant (0.70 mg/ml) was 3 h unfolded in 4 M GdmCl, 18 mM DTT and 1.0 mM EDTA at pH 9.0, the solution was then 25-fold diluted into decreasing GdmCl concentrations and, after 3 h, fluorescence emission spectra were recorded. The functionality of all the mutants refolded under reducing conditions was tested by ELISA (see above).

ii) Data Analysis.

The changes in intrinsic fluorescence emission spectra at increasing GdmCl concentrations were quantified as the intensity-averaged emission wavelengh, X(Roger et al., 1993) calculated according to the following equation:

−λ=Σ(Iiλi)/Σ(Ii) Eq. 1

where λi and Ii are the emission wavelength and its corresponding fluorescence intensity at that wavelength. Baseline and transition region data for scFv(F8) GdmCl denaturation were fit to a two-state (Santoro et al., 1988) linear extrapolation model (LEM) according to the following equation: ΔG_{unfolding}=ΔG^{H}^{₂}^{O}+m_{g} [GdmCl]=−RT lnK_{unfold}. Eq. 2 where ΔG_{unfold.} is the free energy change for unfolding for a given denaturant concentration, ΔG^{H}^{₂}^{O} is the free energy change for unfolding in the absence of denaturant and m is a slope term which quantitates the change in ΔG_{unfolding} per unit concentration of denaturant, R is the gas constant, T is the temperature and K_{unfolding} is the equilibrium constant for unfolding. The model expresses the signal as a function of denaturant concentration:

$${y}_{i} = \frac{{y}_{N} + {{m}_{N} [ x ]}_{i} + ( {y}_{D} + {{m}_{D} [ x ]}_{i} ) * exp [ ( - Δ {G}^{{H}_{2} O} - {{m}_{g} [ x ]}_{i} ) / R T ]}{1 + exp [ ( - Δ {G}^{{H}_{2} O} - {{m}_{g} [ x ]}_{i} ) / R T ]} \qquad \text{Eq. 3}$$

where yᵢ is the observed signal, y_{N} and y_{D} are the baseline intercepts corresponding to native and denatured proteins, respectively, m_{N} and m_{D} are the corresponding baseline slopes, [X]ᵢ is the denaturant concentration after the ith addition, ΔG^{H2O} is the extrapolated free energy of unfolding in the absence of denaturant, mg is the slope of a ΔG unfolding versus [X] plot, R is the gas constant and T is the temperature. The [GdmCl]_{0.5} is the denaturant concentration at the midpoint of the transition and, according to Eq. 2, is calculated as:

[GdmCl]_{0.5}=ΔG^{H}^{O}^{/m}_{g} Eq. 4

f. Affinity Measurements

Affinity-purified scFvG4 and scFvB4 antibodies against the cucumber mosaic virus (CMV) were concentrated to 100 g/ml, assuming that an OD_{280 nm} reading of 1.0 corresponds to an scFv concentration of 0.7 mg/ml. Binding properties were evaluated using surface plasmon resonance (SPR). Real time interaction analysis were performed in BIAcoreX biosensor system (Pharmacia Biosensor AB).

Approximately 5400 resonance units (RU) of purified CMV (200 ng/μl in 7 mM acetate buffer pH 4.0) were coupled to a CM5 sensor chip by using the Amine Coupling kit (Biacore AB) in order to immobilise the antigen. Kinetic analysis wwere performed under continuous flow of 20 μl/min. After each binding measurement, the surface was regenerated with 10 mM HCl. Rate constants were measured on the basis of a single site model using the BIAevaluation 2.1 software (Biacore AB). The association rate constants (kₒₙ) were determined from a plot of ln(dR/dt)/t versus concentration over a range of six concentrations (120, 150, 250, 300, 400 and 450 nM) for both scFvs. The dissociation rate constant (k_{off}) was determined from the dissociation phase in the sensorgram utilizing the following equation:

ln Rt₀/Rt=k_{off}(t−t₀)

where Rt₀ is the response 30 s after completion of antibody injection. A linear plot of ln Rt/Rt₀ vs (t−t₀) yields k_{off} directly as a measurement of the slope. The whole affinity constants (k_{D}) were calculated from the association and dissociation rate constants as k_{D}=k_{off}/kₒₙ. Affinity values were 60 nM for scFvG4 and 10 nM for scFvB4.

### Example 8

### Expression of Proteins in the Plant Cytoplasm

a. Cloning in PVX-Derived Vector and Expression in N. benthamiana Plants

We delivered the scFvs constructs to plants through a potato virus X (PVX)-derived vector (Chapman et al., 1992) in order to obtain high expression levels. Sequences encoding the scFvs were amplified utilizing oligonucleotides designed to insert the recognition sites ClaI, SphI and XbaI upstream (PVX CSX back=TTC ATC GAT TTG CAT GCT CTA GAC ATG CAG GTG CAG CTG CAG) and the recognition site SalI downstream (PVX flag=TCC GTC GAC CTA CTT GTC GTC GTC GTC TCC GTA GTC) the scFv genes. The scFvs genes were then inserted as ClaI-SalI fragments into the polylinker of the vector pPVX201 (Baulcombe et al., 1995), a derivative of pGC3 vector (Chapman et al., 1992), containing unique cloning sites engineered downstream of the PVX coat protein duplicated subgenomic promoter and a CaMV (cauliflower mosaic virus) 35S promoter. Thus, the PVXscFv(G4) and PVXscFv(B4) constructs were obtained. These plasmids could be used directly as inoculum. DNA of each construct (40 μg) was used to mechanically inoculate N. benthamiana plants at the three-four leaf stage, two leaves per plant. Four separate experiments were performed, infecting at least plants with each construct.

Symptoms appeared on the upper leaves usually 7-9 days after inoculation. Symptomatic leaves were frozen in liquid nitrogen and subsequently homogenised in PBS buffer containing a cocktail of protease inhibitor (Complete™, EDTA-free, Boehringer). After centrifugation at 20,000×g for 30 min at 4° C., the supernatants were used to determine total soluble protein (TSP) concentration by using the Bio-Rad Protein assay and BSA as a standard. Western blots were performed using the monoclonal antibody anti-FLAG M2 (Sigma). Immunoblots confirmed the presence of a 30 K molecule, corresponding to scFvB4 or scFvG4, in the soluble fraction.

b. Tomato Transformation

The scFvB4 and scFvG4 genes were subsequently subcloned from the PVXscFv(G4) and PVXscFv(B4) constructs as Xba I-Sal I fragments into a pBI-derived vector under the control of the CaMV 35S promoter. The plasmids were then transferred into the Agrobacterium tumefaciens strain EHA105 by electroporation and used for leaf disc transformation of the miniatur Lycopersicon esculentum cultivar, Micro-Tom (microtomato) (Meissner et al., 1997). Transgenic microtomato plants were regenerated essentially as described (van Roekel et al., 1993).

Trasformation of plants included three independent experiments for both constructs and primary transformants were selected for functional expression. Signals corresponding to the scFvB4 and scFvG4 antibodies were detected both by ELISA and immunoblotting. In particular, the ELISA test was performed according to the following procedure: 5 μg/ml of purified CMV antigens in PBS were coated O/N at 4° C. on a microtitre plate. After blocking with 5% milk in PBS, soluble plant extracts (obtained as described above) were added O/N at 4° C. Functional binding was assessed by using the monoclonal antibody anti-FLAG M2 (2.5 μg/ml) and a rabbit anti-mouse peroxidase-conjugated antibody (KPL). For signal development, ABTS Peroxidase Substrate (KPL) was used. Several transgenic plants expressing the recombinant antibody have been obtained and challanged with the virus.

### BIBLIOGRAPHY

Harlow E., & Lane D. (1988) in Antibodies: a Laboratory Manual (Cold Spring Harbor Laboratory Press, New York.

Kabat E. A., Wu T. T., Perry H. M., Gottesmann K. S., & Foeller C. (1991) ‘Sequences of Proteins of Immunological Interest’. 5^{th} ed. US Department of Health and Human Services, US Government Printing Office.

Neri D., Petrul H., Winter G., Light Y., Marais R., Britton K. E., & Creighton A. M. (1996) in Nature Biotechnology 14: 485-490

Nissim A., Hoogenboom H. R., Tomlison I. M., Flynn G., Midgley C., Lane D. & Winter G. (1994) in The Embo Journal, 13: 692-698.

Sambrook J., Fritsch E. F., & Maniatis T. (1989) in Molecular Cloning (Cold Spring Harbor Laboratory Press, New York).

Tavladoraki P., Benvenuto E., Trinca S., De Martinis D., Cattaneo A., & Galeffi, P. (1993) in Nature 366: 469-472.

Tavladoraki P., Girotti A., Donini M., Arias F. J., Mancini C., Morea V., Chiaraluce R., Consalvi V., & Benvenuto E. (1999) in European Journal of Biochemistry 262: 617-624.

Visintin M., Tse E., Axelson H., Rabbitts T. H., & Cattaneo A. (1999) Proc. Natl. Acad. Sci. 96: 11723-11728.

Bhat T. N. Bentley G. A., Fischman T. O., Boulot G., and Poljak R. G. (1990) in Nature 347: 483-485.

Jung, S. Honegger A. & Plücthun A. (1997). improving in vivo folding and stability of a single chain Fv antibody fragment by loop grafting Protein Eng. 10, 959-966

Baulcombe D. C., Chapman S. & Santa Cruz S. S. (1995). in Plant Journal 7, 1045-1053.

Chapman S., Kavanagh T. & Baulcombe D. C. (1992). in Plant Journal 2, 549-557.

Meissner R., Jacobson Y., Melamed S., Levyatuv S., Shalev G., Ashri A., Elkind Y & Levy A. (1997). in Plant Journal 12, 1465-1472.

Santoro, M. M. & Bolen, D. W. (1988). Unfolding free energy changes determined by the linear extrapolation method. 1. Unfolding of phenylmethanesulfonyl alpha-chymotrypsin using different denaturants. Biochemistry, 27, 8063-8068.

Van Roekel J. S. C., Damm B., Melchers L. S. & Hoekema A. (1993). in Plant Cell Reports 12, 644-647.

1. A peptide characterized
in comprising a sequence selected from the group conisting of the sequences reported in the annexed sequence listing from SEQ ID NO: 1 to SEQ ID N: 8,
and in that
included in a variable region of an antibody make said antibody soluble and stable in cytoplasm,
the peptides comprising the sequences from SEQ ID NO: 1 to SEQ ID NO: 4 being included in the variable region of the heavy chain of an antibody, covalently linked to peptides comprising the sequences reported in the annexed sequence listing from SEQ ID NO: 88 to SEQ ID NO: 90
in the order
SEQ ID NO:1-SEQ ID NO:88-SEQ ID NO:2-SEQ ID NO:89-SEQ ID NO:3-SEQ ID NO:90-SEQ ID NO:4, and
the peptides comprising the sequences from SEQ ID NO: 5 to SEQ ID NO: 8 being included in the variable region of the light chain of an antibody, covalently linked to peptides comprising the sequences reported in the annexed sequence listing from SEQ ID NO: 91 to SEQ ID NO: 93
in the order
SEQ ID NO:5-SEQ ID NO:91-SEQ ID NO:6-SEQ ID NO:92SEQ ID NO:7-SEQ ID NO:93-SEQ ID NO:8:

2. The peptide according to claim 1, wherein
said peptide comprises the sequence reported in the annexed sequence listing as SEQ ID NO: 1, and Xaa in position 24 is Ala;
said peptide comprises the sequence reported in the annexed sequence listing as SEQ ID NO: 2, and Xaa in position 12 is Leu;
said peptide comprises the sequence reported in the annexed sequence listing as SEQ ID NO: 3, and Xaa in position 2 is Pro, Xaa in position 3 is Asp, Xaa in position 13 is Arg, Xaa in position 18 is Asn, and/or Xaa in position 20 is Leu; or
said peptide comprises the sequence reported in the annexed sequence listing as SEQ ID NO: 7, and Xaa in position 12 is Arg, and/or Xaa in position 15, is Phe.

3. A process for preparing a polypeptide suitable as a variable region of an antibody stable and soluble in cytoplasm and specific for a predetermined antigen comprising the step of:
producing an antibody having as a variable region of the heavy chain the polypeptide comprising the sequence reported in the sequence listing as SEQ ID NO: 101, and as a variable region of the light chain the polypeptide comprising the sequence reported in the annexed sequence listing as SEQ ID NO: 102;
putting in contact said antibody with said antigen and
selecting the antibody binding said antigen,
isolating the polypeptide of the variable region of the heavy chain and/or the polypeptide of the variable region of the light chain of the antibody binding said antigen.

4. The process according to claim 3, futher comprising the step of
sequencing the variable regions of said antibody binding said antigen.

5. The process according to claim 3 or 4, wherein said antigen is Tat, Rev, E7 or NS3 protein.

6. A polypeptide comprising the sequence reported in the annexed sequence listing as SEQ ID NO: 101 or SEQ ID NO: 102.

7. A polypeptide obtainable by the process according to claim 3.

8. The polypeptide according to claim 7, wherein said polypeptide comprises a sequence selected from the group consisting of the sequences reported in the sequence listing from SEQ ID NO:31 to SEQ ID NO: 48.

9. A process for producing a peptide conferring to an antibody binding specificity to a predetermined antigen comprising the step of:
producing an antibody having as a variable region of the heavy chain the polypeptide comprising the sequence reported in the sequence listing as SEQ ID NO: 101, and as a variable region of the light chain the polypeptide comprising the sequence reported in the annexed sequence listing as SEQ ID NO: 102;
putting in contact said antibody with said antigen and
selecting the antibody binding said antigen,
isolating the polypeptide of the variable region of the heavy chain and/or the polypeptide of the variable region of the light chain of the antibody binding said antigen;
isolating from said polypeptide the part conferring binding specificity to said antibody.

10. A peptide obtainable by the process according to claim 9.

11. The peptide according to claim 10 characterized by the fact of comprising a sequence selected from the group-consisting of the sequences reported in the annexed sequence listing from SEQ ID NO:9 to SEQ ID NO: 30 and as SEQ ID NO: 94.

12. An antibody characterized by the fact of including as a variable region of the heavy chain or as a variable region of the light chain the polypeptide according to claim 7.

13. An antibody according to claim 12 characterized by the fact of including as variable region of the heavy chain a polypeptide comprising the sequence reported in the annexed sequence listing as SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, or SEQ ID NO: 47, and respectively as variable region of the light chain a polypeptide comprising the sequences reported in the annexed sequence listing as SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, or SEQ ID NO:48.

14. An antibody according to claim 12, wherein said antibody is a scFv, FAB, Fv, dAb, IgG or IgA.

15. An antibody according to claim 13, wherein said antibody is a scFv, FAB, IgG or IgA.

16. An antibody according to claim 14 or 15, wherein said antibody is a scFv and the polypeptides included as variable regions of the heavy chain and of the light chain are connected by a linker.

17. An antibody according to claim 16, wherein said linker comprises the sequence reported in the annexed sequence listing in as SEQ ID NO: 51.

18. A process for obtaining an antibody according to any of claim 12 to 17,
producing an antibody having as a variable region of the heavy chain the polypeptide having the sequence reported in the sequence listing as SEQ ID NO: 101, and as a variable region of the light chain the polypeptide having the sequence reported in the annexed sequence listing as SEQ ID NO: 102;
putting in contact said antibody with said antigen and
selecting the antibody binding said antigen.

19. A polynucleotide characterized by the fact of coding for a peptide according to any one of claims 1, 2, 10, and 11.

20. A polynucleotide characterized by the fact of coding for a polypeptide according to any of claims 6 to 8.

21. A polynucleotide according to claim 20, comprising a sequence selected from the group consisting of sequences reported in the annexed sequence listing as SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 89, SEQ ID NO: 90, SEQ ID NO: 91, SEQ ID NO: 92, SEQ ID NO: 93, SEQ ID NO: 94 and SEQ ID NO: 95.

22. A polynucleotide characterized by the fact of coding for an antibody according to any one of claims 12 to 17.

23. A polynucleotide according to claim 22, comprising a sequence selected from the group consisting of sequences reported in the annexed sequence listing as SEQ ID NO: 84 and SEQ ID NO: 69.

24. A pharmaceutical composition characterized by the fact of including as an active agent a therapeutically effective amount of an antibody according to any one of claims 12 to 17 together with a pharmaceutically acceptable carrier vehicle or auxiliary agent.

25. A pharmaceutical composition characterized by the fact of including as an active agent a therapeutically effective amount of the polynucleotides according to any one of claims 19 to 23 together with a pharmaceutically acceptable carrier vehicle or auxiliary agent.

26. The antibody according to any one of claims 12 to 17, for use as a medicament.

27. Use of the antibody according to any one of claims 12 to 17, for the manufacture of a medicament for the treatment of pathologies associated with accumulation of a molecule inside or outside a human, or animal cell.

28. A polynucleotide according to any one of the claims 25 to 29, for use as a medicament.

29. Use of the polynucleotide according to any one of the claims 19 to 23, for the manufacture of a medicament for the gene therapy of pathologies associated with the accumulation of a molecule inside or outside a human or animal cell.

30. Use of the antibodies according to any one of claims 12 to 17, and/or of the polynucleotides according to any one of claims 19 to 23, for the diagnosis of pathologies associated with the accumulation of a molecule inside or outside a human, animal or plant cell.

31. A diagnostic kit comprising as a reagent an antibody according to any of claims 12 to 17, and/or the polynucleotides according to any one of claims 19 to 23.

32. A diagnostic kit comprising as a reagent a peptide according to claim 10 or 11.

33. Use of an antibody according to any of claims 12 to 17 and/or of a polynucleotide according to claim 19 to 23 for the treatment of pathologies associated with the accumulation of a molecule inside or outside a plant cell.
